(19) United States
(12) Patent Application Publication
(10) Pub. No.: US 2012/0058208 A1
(43) Pub. Date: Mar. 8, 2012

(54) Synergistic Composition for Enhancing Bioavailability of Curcumin

(75) Inventor: Chennakattu Varkey Jacob, Kochi (IN)

(73) Assignee: SYNTHITE INDUSTRIES LTD., Kochi (IN)

(21) Appl. No.: 13/083,388

(22) Filed: Apr. 8, 2011

(30) Foreign Application Priority Data
Sep. 4, 2010 (IN) 999/CHE/2010

(51) Int. Cl. A61K 36/906; A61K 31/165; A61P 35/00; A61K 36/898

(52) U.S. Cl. 424/756; 424/725; 514/627

(57) ABSTRACT

The present disclosure relates to a composition to enhance the bioavailability of curcumin. In one embodiment, a composition comprising plant extracts of curcumin, vanilla and ginger, wherein the extracts of ginger and vanilla are rich in gingerol and vanillin respectively, is provided. In other embodiments, curcumin, and one or more items selected from the group of vanilla, ginger and capsaicin is provided.

### CROSS-REFERENCE TO RELATED APPLICATION

This application claims priority to and the benefit of Indian Provisional Patent Application number 999/CHE/2010, entitled “A Synergistic Composition for Enhancing Bioavailability of Curcumin,” filed Apr. 9, 2010, the contents of which are hereby incorporated by reference into the present application.

### BACKGROUND

1. Technical Field

The present disclosure relates generally to a composition and related methods for providing health benefits, and, more particularly, to compositions and related methods of ginger and vanilla extract with curcumin to increase the bioavailability of curcumin and inhibiting tumor growth.

2. Description of Related Art

Research in the latter half of the 20th century has identified that curcumin has a wide range of potential therapeutic and preventive effects. So far, these effects have not been confirmed in humans. However, as of 2008, numerous clinical trials in humans were underway, studying the effect of curcumin on numerous diseases including multiple myeloma, pancreatic cancer, myelodysplastic syndromes, colon cancer, psoriasis, and Alzheimer's disease [1].

In vitro and animal studies have suggested the curcumin may have antitumor [2] [3], antioxidant, anti-arthritic, anti-amyloid, anti-ischemic [4], and anti-inflammatory properties [5]. Anti-inflammatory properties may be due to inhibition of eicosanoid biosynthesis [6]. In addition, curcumin may be effective in treating malaria, preventing cervical cancer, and may interfere with the replication of the HIV virus [7]. In HIV, curcumin appears to act by interfering with the P300/CREB-binding protein (CBP). It is also hepatoprotective [8].

A 2008 study at Michigan State University showed that low concentrations of curcumin interfere with Herpes Simplex Virus-1 (HSV-1) replication [9]. The same study showed that curcumin inhibited the recruitment of RNA polymerase II to viral DNA, thus inhibiting the transcription of the viral DNA. This effect was shown to be independent of the effect on the histone acetyltransferase activities of p300/CBP. A 1999 University of Cincinnati study indicated that curcumin is significantly associated with protection from infection by HSV-2 in animal models of intravaginal infections [10].

Curcumin acts as a free radical scavenger and antioxidant, inhibiting lipid peroxidation [11] and oxidative DNA damage. Curcuminoids induce glutathione S-transferase and are potent inhibitors of cytochrome P450. A 2004 UCLA-Veterans Affairs study involving genetically altered mice suggested that curcumin might inhibit the accumulation of destructive beta-amyloid in the brains of Alzheimer's disease patients and also break up existing plaques associated with the disease [12].

There is also circumstantial evidence that curcumin improves mental functions. A survey of 1,010 Asians who ate yellow curry and were between the ages of 60 and 93 showed that those who ate the sauce “once every six months” or more had higher MMSE results than those who did not [13]. From a scientific standpoint, though, this does not show whether the curry caused it, or people who had healthy habits also tended to eat the curry, or some completely different relationship.

Numerous studies have demonstrated that curcumin, amongst only a few other things such as high impact exercise, learning, bright light, and antidepressant usage, has a positive effect on neurogenesis in the hippocampus and concentrations of brain-derived neurotrophic factor (BDNF), reductions in both of which are associated with stress, depression, and anxiety [14] [15] [16]. Curcumin has also been demonstrated to be a selective monoamine oxidase inhibitor (MAOI) of type MAO-A.

In 2009, an Iranian group demonstrated the combination effect of curcumin with 24 antibiotics against Staphylococcus aureus. It was shown that in the presence of a sub-inhibitory concentration of curcumin, the antibacterial activities of cefixime, cefotaxime, vancomycin and tetracycline increased against test strain [17]. Curcumin's potential anticancer effects stem from its ability to induce apoptosis in cancer cells without cytotoxic effects on healthy cells. Curcumin can interfere with the activity of the transcription factor NF-κB, which has been linked to a number of inflammatory diseases such as cancer [18].

A 2009 study suggested that curcumin may inhibit mTOR complex I via a novel mechanism [19]. Another 2009 study on curcumin effects on cancer stated that curcumim “modulates growth of tumor cells through regulation of multiple cell signaling pathways including cell proliferation pathway (cyclin D1, c-myc), cell survival pathway (Bcl-2, Bcl-xL, cFLIP, XIAP, c-IAP1), caspase activation pathway (caspase-8,3,9), tumor suppressor pathway (p53, p21) death receptor pathway (DR4, DR5), mitochondrial pathways, and protein kinase pathway (JNK, Akt, and AMPK)” [20]. When 0.2% curcumin was added to a diet given to rats or mice previously given a carcinogen, it significantly reduced colon carcinogenesis.

The pharmacological safety and efficacy of curcumin makes it a potential compound for the treatment and prevention of a wide variety of human diseases. In spite of its efficacy and safety, curcumin has not yet been approved as a therapeutic agent, and the relative bioavailability of curcumin has been highlighted as a major problem for this. Major reasons contributing to the low plasma and tissue levels of curcumin appear to be due to poor absorption, rapid metabolism, and rapid systemic elimination. The low serum level of curcumin is one of the major factors affecting its bioavailability. A recent study showed that after oral administration of 400 mg of curcumin to rats, only traces of unchanged drug were found in the liver and kidney. At 30 min, 90% of curcumin was found in the stomach and small intestine, but only 1% was present at 24 hours. Once absorbed, curcumin is metabolized rapidly in the liver and excreted out of the biological system. This seriously limits the ability of curcumin to reach targets distant from the gut and exert its beneficial action.

To improve the bioavailability of curcumin, numerous approaches have been undertaken. The reports so far show that the absorption, biodistribution, metabolism and elimination of curcumin are the major problems that affect the bioavailaibility of curcumin. Several strategies has been tried to overcome these problems. The use of adjuvants (bioavailability enhancers) is one important means in this route. Other methods include nanoparticle technology, encapsulation of curcumin in liposome, complexation of curcumin to form micelles and phospholipids, and the introduction of bioconjugates.

Adjuvants are compounds that are concomitantly administered with the drug/nutraceutical so as to enhance the bioavailability of the latter. One of the main reasons for the poor bioavailability of curcumin is glucuronidation in the liver. The adjuvants like piperine are shown to inhibit this metabolic pathway of glucuronidation, thus increasing the bioavailability of curcumin. There are several reports of other adjuvants that show synergetic effects when used in combination with curcumin.

Several adjuvants are reported to enhance the bioavailability of curcumin. Concomitant administration of piperine along with curcumin (Bioperin) has been shown to increase the bioavailability of curcumin. Piperine inhibits hepatic and intestinal glucuronidation, which is the major pathway of curcumin metabolism, thereby reducing glucuronidation of curcumin, and subsequently reducing the elimination from the body. The human body uses glucuronidation to make a large variety of substances more water-soluble, and, in this way, allows for their subsequent elimination from the body upon urination.

Gingerol has also been reported to enhance the bioavailability of curcumin. In a patent work by Khajuria et el (2003), it was shown that gingerol enhances the bioavailability of several nutraceutical drugs such as vitamins A, E, C, Folic acid, b-carotene, silymarin, isoleucine, zinc and potassium. The patent shows a detailed study of gingerol use as a bioavailability enhancer. A comparison of the activity of gingerol to piperine and a combination of (50:50) gingerol: piperine is also reported. For the given dose (50 mg/kg) gingerol was shown to enhance the bioavailability of curcumin by 43% whereas piperine showed the same enhancement by 33%. A combination of gingerol and piperin showed a curcumin bioenhancement of 70%. The screening and comparison has been made for several immunosuppresents, herbal formulations, cortisosteroids, anti-histamines and anti-ulcer drugs, which shows gingerol has a higher bioenhancing activity when compared to piperine. Other reported bioavailability enhancers of curcumin are eugenol, EGCG, quarecetin and genisetein. The activity of these adjuvants is not as effective as that of piperine and gingerol.

### REFERENCES

1. Hatcher H, Planalp R, Cho J, Torti F M, Torti S V (June 2008). “Curcumin: from ancient medicine to current clinical trials”. Cell. Mol. Life. Sci. 65 (11): 1631-52.
2. Aggarwal, B B.; Shishodia S. (May 2006). “Molecular targets of dietary agents for prevention and therapy of cancer”. Biochemical Pharmacology (Elsevier) 71 (10): 1397-421.
3. Choi, Hyunsung; et al. (July 2006). “Curcumin Inhibits Hypoxia-Inducible Factor-1 by Degrading Aryl Hydrocarbon Receptor Nuclear Translocator: A Mechanism of Tumor Growth Inhibition”. Molecular Pharmacology (American Society for Pharmacology and Experimental Therapeutics) 70 (5): 1664-71.
4. Shukla P K, Khanna V K, Ali M M, Khan M Y, Srimal R C. “Anti-ischemic effect of curcumin in rat brain”, Neurochem Res. 2008 June; 33(6):1036-43.
5. Stix, Gary (February 2007). “Spice Healer”. Scientific American.
6. Srivastava, K C; Bordia A; Verma S K (April 1995). “Curcumin, a major component of the food spice turmeric (Curcuma longa), inhibits aggregation and alters eicosanoid metabolism in human blood platelets”. Prostaglandins Leukot Essent Fatty Acids 52 (4): 223-7.
7. Padma, T V (2005-03-11). “Turmeric can combat malaria, cancer virus and HIV”. SciDev.net.
8. Marotta, F.; et al. (October 2003). “Hepatoprotective effect of a curcumin/absinthium compound in experimental severe liver injury”. Chinese Journal of Digestive Diseases (Blackwell Publishing) 4 (3): 122-7.
9. Kutluay S B, Doroghazi J, Roemer M E, Triezenberg S J (January 2008). “Curcumin inhibits herpes simplex virus immediate-early gene expression by a mechanism independent of p300/CBP histone acetyltransferase activity”. Virology 373 (2): 239.
10. Bourne K Z, Bourne N, Reising S F, Stanberry L R (1999 July). “Plant products as topical microbicide candidates: assessment of in vitro and in vivo activity against herpes simplex virus type 2”. Antiviral research 42 (3): 219-26.
11. Shukla P K, Khanna V K, Khan M Y, Srimal R C. “Protective effect of curcumin against lead neurotoxicity in rat”. Hum Exp Toxicol. 2003 December; 22(12):653-8.
12. Yang, F; Lim G P; Begum A N; Ubeda O J; Simmons M R; Ambegaokar S S; Chen P P; Kayed R; Glabe C G; Frautschy S A; Cole G M (February 2005). “Curcumin inhibits formation of amyloid beta oligomers and fibrils, binds plaques, and reduces amyloid in vivo”. Journal of Biological Chemistry (American Society for Biochemistry and Molecular Biology) 280 (7): 5892-901.
13. Ng T P; Chiam P C; Lee T; Chua H C; Lim L; Kua E H (November 2006). “Curry consumption and cognitive function in the elderly”. American Journal of Epidemiology (Oxford University Press) 164 (9): 898-906.
14. Xu Y, Ku B, Cui L, Li X, Barish P A, Foster T C, Ogle W O. (August 2007). “Curcumin reverses impaired hippocampal neurogenesis and increases serotonin receptor 1A mRNA and brain-derived neurotrophic factor expression in chronically stressed rats”. Brain Res 1162: 9.
15. Wu A, Ying Z, Gomez-Pinilla F. (February 2006). “Dietary curcumin counteracts the outcome of traumatic brain injury on oxidative stress, synaptic plasticity, and cognition”. Experimental Neurology 197 (2): 309.
16. Bala K, Tripathy B C, Sharma D. (April 2006). “Neuroprotective and anti-ageing effects of curcumin in aged rat brain regions”. Biogerontology 7 (2): 81.
17. Kamyar Mollazadeh Moghaddam, Mehrdad Iranshahi, Mahsa Chitsazian Yazdi, Ahmad Reza Shahverdi (August 2009). “The combination effect of curcumin with different antibiotics against Staphylococcus aureus”. IJGP 3(2):141-143.
18. Aggarwal B B, Shishodia S. “Suppression of the nuclear factor-kappaB activation pathway by spice-derived phytochemicals: reasoning for seasoning”. Ann N Y Acad. Sci. 2004 December; 1030:434-41.
19. Beevers C S, Chen L, Liu L, Luo Y, Webster N J, Huang S (February 2009). “Curcumin disrupts the Mammalian target of rapamycin-raptor complex”. Cancer Res. 69 (3): 1000-8.
20. Ravindran, J.; Prasad, S.; Aggarwal, B. (July 2009). “Curcumin and Cancer Cells: How Many Ways Can Curry Kill Tumor Cells Selectively?”. The AAPS journal 11 (3): 495.

### SUMMARY

In one embodiment of the present disclosure, a synergistic composition for enhancing the bioavailability of curcumin, containing curcumin, vanilla extract and ginger extract, optionally along with pharmaceutically acceptable excipients is provided and a related method of providing bioaviable curcumin is also provided. In another embodiment of the present disclosure, a process for preparation of a synergistic composition for enhancing the bioavailability of curcumin containing curcumin, vanilla extract and ginger extract comprising acts of, mixing the curcumin, vanilla extract and ginger extract to obtain a mixture, blending the mixture, and optionally adding excipients to obtain the synergistic composition is provided. In yet another embodiment of the present disclosure, a method of enhancing the bioavailability of curcumin, the method including administering biologically suitable amounts of a synergistic composition including curcumin, vanilla extract and ginger extract, optionally along with pharmaceutically acceptable excipients to a subject in need thereof, is provided.

In yet another embodiment of the present disclosure, a synergistic composition for enhancing the bioavailability of curcumin, containing curcumin, vanilla extract and ginger extract, optionally along with pharmaceutically acceptable excipients, is provided.

In yet another embodiment of the present disclosure, a synergistic composition for enhancing the bioavailability of curcumin, containing curcumin and vanilla extract and/or capsaicin optionally along with pharmaceutically acceptable excipients, is provided

In an aspect of at least one embodiment of the present disclosure, curcumin is present in a range from about 60% weight/weight to about 85% weight/weight, ginger extract is present in a range from about 5% weight/weight to about 25% weight/weight, and the vanilla extract is present in a range from about 0.5% weight/weight to about 5% weight/weight.

In another aspect of at least one embodiment of the present disclosure, curcumin is present in a range of about 84% w/w to about 86% w/w, ginger extract is present in a range of about 12% w/w to about 13% w/w and vanilla extract is present in a range of about 2% w/w to about 3% w/w

In another aspect of at least one embodiment of the present disclosure, curcumin contains curcuminoids in a range of about 92% to a range of about 94%, ginger extract contains gingerol in a range of about 10% to a range of about 12% and vanilla extract contains vanillin in a range of about 0.5% to a range of about 2.5%.

In yet another aspect of at least one embodiment of the present disclosure, the curcumin, vanillin and gingerol can be obtained from one or more plants and by chemical synthesis.

In yet another aspect of at least one embodiment of the present disclosure, the excipients are selected from a group consisting of granulating agents, binding agents, lubricating agents, disintegrating agents, sweetening agents, glidants, anti-adherents, anti-static agents, surfactants, antioxidants, coating agents, coloring agents, flavoring agents, plasticizers, preservatives, suspending agents, emulsifying agents and spheronization agents.

In yet another aspect of at least one embodiment of the present disclosure, the composition is formulated into dosage forms selected from a group consisting of tablets, troches, lozenges, aqueous or oily suspensions, capsule, emulsion, creams, sprays, drops, dispersible powders or granules, syrups, elixirs, phytoceuticals, nutraceuticals and food stuffs.

In yet another embodiment of the present disclosure, a process is disclosed for the preparation of a synergistic composition for enhancing the bioavailability of curcumin containing curcumin, vanilla extract and ginger extract including, but not limited to, mixing the curcumin, the vanilla extract and the ginger extract to obtain a mixture, blending the mixture, and optionally adding excipients to obtain the synergistic composition.

In yet another aspect of at least one embodiment of the present disclosure, the curcumin, vanilla extract and ginger extract are in powder form.

In yet another aspect of at least one embodiment of the present disclosure, the blending is carried out using a blender selected from a group consisting of a ball mill mixer, sand mill mixer, multi mill mixer, pulverizer, and granulator.

In yet another embodiment of the present disclosure, a method is disclosed of enhancing the bioavailability of curcumin. In an aspect of at least one embodiment, the method involves administering biologically suitable amounts of a synergistic composition comprising curcumin, vanilla extract and ginger extract, optionally along with pharmaceutically acceptable excipients to a subject in need thereof.

In another aspect of at least one embodiment of the present disclosure, the subject is a mammal. In another aspect of at least one embodiment, the subject is a human.

One embodiment of the present disclosure provides use of a combination of ginger extract rich in gingerol (up to 35% purity) and vanilla extract rich in vanillin (up to 5% purity) that enhances the bioavailability of curcumin. It was found that when a composition (composition 1) containing ginger extract rich in gingerol (10%) and vanilla extract (0.75% rich in vanillin) with curcumin (94% curcuminoids) is administered orally to Sprague dawley rats, the concentration of curcumin in blood plasma had a significant increase when compared to the control. The enhancement in bioavailability is found to be about 7.5 folds when compared to the control. There is an enhancement in the bioavailability of the curcumin when the composition is used as compared to the bioavailability of curcumin when used with ginger extract and/or vanilla extract per se.

Apart from enhancing the bioavailability of curcumin with the help of the extract, the composition smells pleasant due to the presence of vanillin.

In yet another embodiment of the present disclosure, the synergistic composition of the present disclosure is also noted for enhancing anti-cancer activity by inhibiting tumor growth.

In yet another embodiment of the present disclosure, a method of reducing the size of one or more tumors in a mammal is provided. The method comprises administering to a mammal having a tumor a composition containing an effective amount of curcumin and an effective amount of ginger extract and an effective amount of vanilla extract.

These, as well as other components, steps, features, objects, benefits, and advantages, will now become clear from a review of the following detailed description of illustrative embodiments, the accompanying drawings, and the claims

### BRIEF DESCRIPTION OF DRAWINGS

The drawings disclose illustrative embodiments. They do not set forth all embodiments. Other embodiments may be used in addition or instead. Details that may be apparent or unnecessary may be omitted to save space or for more effective illustration. Conversely, some embodiments may be practiced without all of the details that are disclosed. When the same numeral appears in different drawings, it is intended to refer to the same or like components or steps.

FIG. 1 is a graph showing the results of oral pharmacokinetics studies in male Sprague Dawley rats (1 g/Kg body weight) demonstrating some of the benefits and advantages of the present disclosure.

FIG. 2 is a graph showing in vivo anti-cancer activity of curcumin composition 1 in an HCT-116 human xenograft model.

### DETAILED DESCRIPTION OF ILLUSTRATIVE EMBODIMENTS

Illustrative embodiments are now discussed. Other embodiments may be used in addition or instead. Details that may be apparent or unnecessary may be omitted to save space or for a more effective presentation. Conversely, some embodiments may be practiced without all of the details that are disclosed.

The details of a method of preparing the composition are given below. However, it should not be construed that the scope disclosure is limited to the examples.

Experiment I: Preparation of Compositions

Materials Used to Prepare Compositions

Curcumin: the turmuric extract with total curcuminoids of 80-94%. Curcumin powder is extracted from turmeric rhizomes (Curcuma longa) using ethyl acetate as a solvent. The extracted oleoresin is refluxed with a non-polar solvent (hexane) for removing the oil content present in the curcumin extract. Crystallizing from a suitable mixture of ethyl acetate and hexane results in further purification. The final crystallization is done using a suitable alcohol like isoproplyl, isobutyl, or neopentyl alcohol to get the total curcuminoids to 94% [Curcumin, demethoxycurcumin (DMC) and bisdemethoxycurcumin (BDMC)]. Hereafter, curcumin is defined as the turmeric extract with total curcuminoids of 92-94%.

Ginger extract: the ginger extract rich in gingerol. Ginger raw material (RM) of the Burma variety is used for supercritical fluid extraction using CO₂ (SCF—CO₂) after sun drying. The RM is loaded into the extractor where the extraction is done for three hours at a bar pressure of 250 and 45° C. The top note is collected in the separator and gingerol-rich ginger extract is collected separately. The ginger extract is centrifuged to remove any moisture content that is in resin form, having a gingerol content of 35%. The ginger extract is mixed with maltodextrin and spray-dried to get a powdered product with a gingerol content of 10% to 12%. Hereafter, ginger extract is defined as the ginger extract mixed with maltodextrin, in the powder form with a gingerol content of 10-12%.

Vanilla extract: the plant extract obtained from grade A vanilla beans (RM) from Karnataka. The RM (having a moisture content of 30%) is extracted in aqueous alcohol. The vanilla extract obtained is mixed with maltodextrin and spray-dried to a powder product with 0.75% vanillin content. Hereafter, vanilla extract is defined as the vanilla plant extract mixed with maltodextrin, in the powder form with a vanillin content of 0.75%.

In an embodiment of the present disclosure, commercially available curcumin, ginger extract and vanilla extract with appropriate activity can also be used.

Composition 1

42.5 grams of curcumin are mixed with 6.25 grams of ginger extract and 1.25 grams of vanilla extract in a mortar and mixed in a lab homogenizer to equal 50 grams of Composition 1.

This formulation is also prepared on a large scale in the plant level. For example, 85 kilgrams of curcumin extract are blended along with 12.5 kilograms of ginger extract and 2.5 kilograms of vanilla extract in a homogenizer to equal 100 kilograms of Composition 1.

Composition 2

42.5 grams of curcumin are mixed with 7.5 grams of ginger extract in a mortar and mixed in a lab homogenizer to equal 50 grams of Composition 2.

Composition 3

44.5 grams of curcumin are mixed with 4.25 grams of ginger extract and 1.25 grams of vanilla extract in a mortar and mixed in a lab homogenizer to equal 50 grams of Composition 3.

Composition 4

46.5 grams of curcumin are mixed with 2.25 grams of ginger extract and 1.25 grams of vanilla extract in a mortar and mixed in a lab homogenizer to equal 50 grams of Composition 4.

Composition 5

43.25 grams of curcumin are mixed with 6.25 grams of ginger extract and 0.5 gram of vanilla extract in a mortar and mixed in a lab homogenizer to equal 50 grams of Composition 5.

Composition 6

49 grams of curcumin are mixed with one gram of vanilla extract in a mortar and mixed in a lab homogenizer to equal 50 grams of Composition 6.

Composition 7

49.5 grams of curcumin are mixed with 0.5 gram of high pure capsaicin in a mortar and mixed in a lab homogenizer to equal 50 grams of Composition 7.

Table I—Compositions screened for oral pharmacokinetics studies

Curcumin

Control

Curcumin 85%+Ginger Extract 12.5%+Vanilla extract 2.5% Composition 1

Curcumin 85%+Ginger Extract 15% Composition 2

Curcumin 89%+Ginger extract 8.5%+Vanilla extract 2.5% Composition 3

Curcumin 93%+Ginger extract 4.5%+Vanilla extract 2.5% Composition 4

Curcumin 86.5%+Ginger extract 12.5%+Vanilla extract 1% Composition 5

Curcumin 98%+Vanilla extract 2% Composition 6

Curcumin 99%+Capsaicin 1% Composition 7

TABLE II
The actual content of active ingredients in compositions by HPLC

| | Composition | | | | | | | |
|---|---|---|---|---|---|---|---|---|
| Ingredient | Control | 1 | 2 | 3 | 4 | 5 | 6 | 7 |
| Curcuminoids % | 94 | 80 | 79.9 | 83.69 | 85 | 81.45 | 92.3 | 93.06 |
| Gingerol % | | 1.5 | 5.3 | 0.77 | 0.51 | 1.45 | | |
| Vanillin % | | 0.04 | | 0.043 | 0.021 | 0.009 | 0.035 | |
| Capsaicin % | | | | | | | | 1.1 |

Formulation

Composition 1 is filled as such into capsules (500 mg each) for oral consumption. In another embodiment of the present disclosure, the compositions are also obtained by mixing appropriate quantities of synthetic curcuminoids, gingerol and vanillin.

Experiment II: Animal Testing

Researchers used Sprague Dawley rats aged 7 to 8 weeks and weighing around 200 to 250 grams. Animals fasted overnight with free access to water. The rats were given the test substance orally with a curcuminoids equivalent dose of 1 g/kg body weight (in a suitable formulation and dose volume). Blood samples (150-200 ml) were collected at various time points during the next 24 hours (0.08, 0.25, 0.5, 1, 2, 4, 8 and 24 hours).

The blood samples were centrifuged at 3000 grams for five minutes at 4° C. and the corresponding plasma samples were harvested in clean, pre-labeled tubes. Analysis by ultra-fast liquid chromatography (UFLC) was carried out on same day, or samples were stored at −80° C. until analysis was performed.

Bioanalytical Procedure

Curcumin in plasma samples quantified using UFLC with suitable extraction and recovery methods.

Data Analysis and Report

The data was analyzed by WinNonlin (Pharsight) to calculate PK parameters: _{AUCO-t}, AUC_{0-∞}, Cₘₐₓ, Tₘₐₓ, t_{1/2} and MRTₗₐₛₜ. The data is summarized in Table III.

TABLE III
Curcumin compositions: Oral pharmacokinetics studies in male Sprague dawley rats (1 g/kg body
weight equivalent dose)
Mean Plasma PK Parameters

| | | Curcumin | | Curcumin | Curcumin | | | |
|---|---|---|---|---|---|---|---|---|
| | | 85% + | | (89%) + | (93%) + | Curcumin | | |
| | | Ginger | | Ginger | Ginger | (86.5%) + | | |
| | | extract | Curcumin | extract | extract | Ginger | Curcumin | |
| | | (12.5%) + | (85%) + | (8.5%) + | (4.5%) + | extract | (98%) + | Curcumin |
| | | Vanilla | Ginger | Vanilla | Vanilla | (12.5%) + | Vanilla | (99%) + |
| | | extarct | extarct | extract | extract | Vanilla | extarct | capsaicin |
| Parameters | Curcumin | (2.5%) | 15% | (2.5%) | (2.5%) | extract (1%) | (2%) | (1%) |
| Composition | Control | 1 | 2 | 3 | 4 | 5 | 6 | 7 |
| number | | | | | | | | |
| Route of | Oral | Oral | Oral | Oral | Oral | Oral | Oral | Oral |
| administration | | | | | | | | |
| Dose (g/kg) | 1 | 1 | 1 | 1 | 1 | 1 | 1 | 1 |
| equivalent dose | | | | | | | | |
| Cmax (ng/mL) | 127 | 290 | 290 | 167 | 143 | 216 | 152 | 149 |
| Tmax (h) | 0.08 | 1 | 0.08 | 2 | 1 | 1.2 | 2 | 0.5 |
| AUClast | 146 | 1104 | 566 | 581 | 321 | 423 | 615 | 498 |
| (h * ng/mL) (0 | | | | | | | | |
| to 24 h) | | | | | | | | |
| AUCinf | 278 | 1372 | 856 | 818 | 367 | 450 | 909 | 966 |
| (h * ng/mL) | | | | | | | | |
| T½ (h) | 1.8 | 2.1 | 3.4 | 5.2 | 1.9 | 1.4 | 4.9 | 7.6 |
| MRT last (h) | 0.83 | 2.8 | 2.29 | 3 | 2.2 | 2.1 | 3.3 | 3.2 |

Results

From the studies above, including but not limited to the results of the studies shown in Table III, it is evident that the presence of ginger extract, vanilla extract and capsaicin enhances the plasma exposure of curcumin.

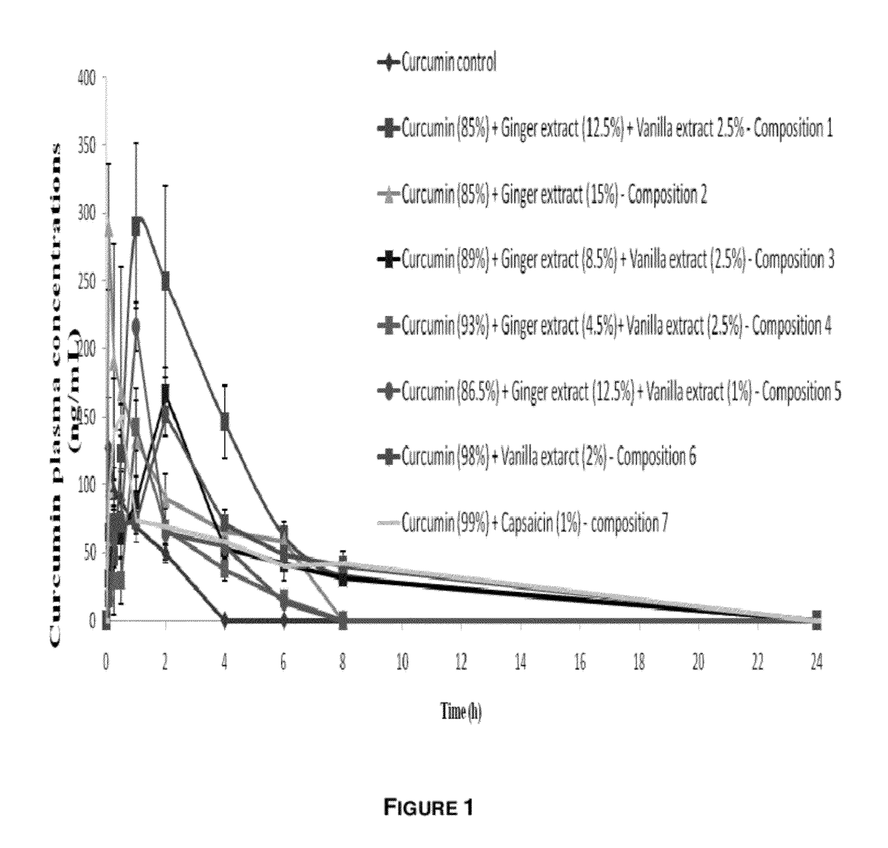

As compared to the curcumin control, the composition of curcumin (85%)+ginger extract (12.5%)+vanilla extract (2.5%) (Composition 1) resulted in a 7.5-fold increase in area under the curve (AUCₗₐₛₜ) and a 2.3 fold increase in Cₘₐₓ as detailed in FIG. 1.

As compared to curcumin (85%)+ginger extract (15%) (Composition 2), the composition of curcumin (85%)+ginger extract (12.5%)+vanilla extract (2.5%) (Composition 1) resulted in a two-fold increase in area under the curve (AUCₗₐₛₜ).

Curcumin+vanilla extract (Composition 6) and curcumin+capsaicin (Composition 7) also showed increased bioavailability as compared to curcumin control.

It should be appreciated that compositions containing one or more items selected from the group consisting of vanilla extract, capsaicin and ginger extract combined with and curcumin worked synergistically to provide increased bioavailability that was statistically significant and far more than the sum of the bioavailability provided by the sum of the various component parts.

Experiment III: Anti-Cancer Activity of Curcumin Composition 1

Human colorectal (HCT-116) xenograft model in SCID mice

Five-week-old female mice purchased from the Advanced Centre for Treatment, Research and Education in Cancer (ACTREC) were used for in vivo experiments. The Institutional Animal Ethics Committee (IAEC) approved the in vivo study. Animals were injected with 1×10⁶ HCT-116 cells subcutaneously in the flank region. Animals were monitored daily during the period between inoculation and palpable tumor growth. Tumor size was measured with a digital Vernier caliper and tumor-bearing mice were randomized into control and treatment groups (n=8), when the tumor volume was attained ˜100 mm³. The tumor-bearing mice were orally administered with the curcumin Composition 1, curcumin control and reference compound (Vorinostat) at the doses mentioned once daily for 28 days. Tumor volume and was body weight was measured twice weekly. The following formula was used to calculate the tumor volume: Tumor volume=(length×width)/2. Percent reduction in tumor growth was calculated with respect to the Vehicle treated group.

TABLE 4
In vivo anti-cancer activity of curcumin Composition 1 in HCT-116 human
xenograft model
Tumor volume (mm³), Mean, ± SE

| Group | Day 0 | Day 4 | Day 8 | Day 12 | Day 16 |
|---|---|---|---|---|---|
| Control | 137.9 ± 25.9 | 235.0 ± 49.2 | 436.5 ± 97.8 | 595.9 ± 115.2 | 768.8 ± 140.0 |
| (Vehicle) | | | | | |
| Vorinostat, | 131.6 ± 23.1 | 236.6 ± 42.7 | 334.4 ± 52.3 | 460.6 ± 76.0 | 544.6 ± 79.1 |
| 150 mg/kg | | | | | |
| Curcumin | 132.3 ± 23.3 | 287.7 ± 52.3 | 405.9 ± 64.0 | 619.5 ± 73.2 | 861.8 ± 147.1 |
| control | | | | | |
| 500 mg/kg | | | | | |
| Curcumin | 136.2 ± 24.1 | 243.4 ± 35.1 | 362.3 ± 46.3 | 516.9 ± 64.3 | 737.3 ± 98.2 |
| Composition 1, | | | | | |
| 500 mg/kg | | | | | |

| | | | | | % |
|---|---|---|---|---|---|
| Group | Day 19 | Day 22 | Day 26 | Day 29 | Redn. |
| Control | 1144.7 ± 186.3 | 1102.3 ± 194.5 | 1445.5 ± 272.2 | 1733.4 ± 325.0 | |
| (Vehicle) | | | | | |
| Vorinostat, | 669.6 ± 107.0 | 726.0 ± 120.8 | 916.6 ± 157.9 | 979.1 ± 157.2 | 47 |
| 150 mg/kg | | | | | |
| Curcumin | 977.4 ± 162.7 | 1052.1 ± 215.0 | 1147.1 ± 245.0 | 1290.2 ± 242.2 | 27 |
| control | | | | | |
| 500 mg/kg | | | | | |
| Curcumin | 832.5 ± 103.1 | 890.4 ± 117.4 | 1021.8 ± 157.5 | 1161.3 ± 214.6 | 36 |
| Composition 1, | | | | | |
| 500 mg/kg | | | | | |

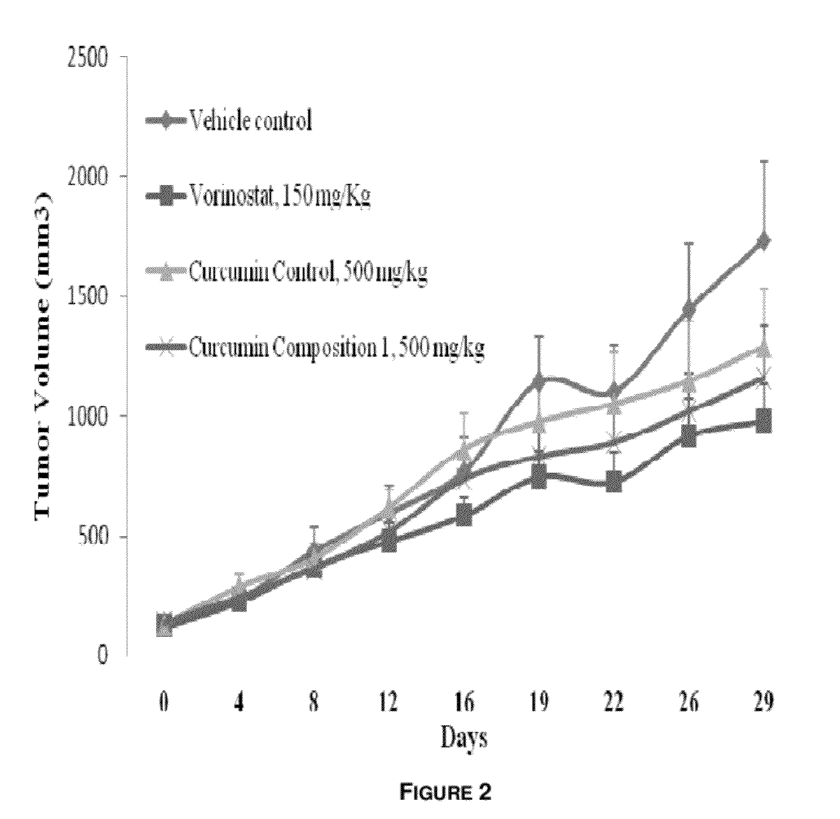

It can be noted that the curcumin Composition 1 evaluated for in vivo anti-cancer activity in an HCT-116 xenograft model of colorectal cancer in SCID mice showed promising results for anti-cancer activity. The formulation at 500 mg/kg dose resulted in a 36% inhibition in tumor growth, compared to 27% with the curcumin control after 28 days of treatment. No significant change was observed in their body weight after 28 days of treatment. FIG. 2 provides the details of the analysis explained above.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The components, steps, features, objects, benefits and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits and advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

Nothing that has been stated or illustrated is intended or should be interpreted to cause a dedication of any component, step, feature, object, benefit, advantage, or equivalent to the public, regardless of whether it is recited in the claims.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

1. A composition for providing bioavailable curcumin, the composition comprising:
an effective amount of curcumin;
an effective amount of ginger extract; and
an effective amount of vanilla extract.

2. The composition of claim 1, wherein the effective amount of curcumin is from about 60% weight/weight to about 85% weight/weight.

3. The composition of claim 1, wherein the effective amount of ginger extract is from about 5% weight/weight to about 25% weight/weight.

4. The composition of claim 1, wherein the effective amount of vanilla extract is from about 0.5% weight/weight to about 5% weight/weight.

5. The composition of claim 1, wherein the effective amount of Curcumin is about 84% w/w to about 86% w/w.

6. The composition as in claim 1 wherein the effective amount of Ginger extract is about 12% w/w to about 13% w/w

7. The composition as in claim 1 wherein the effective amount of Vanilla extract is about 2% w/w to about 3% w/w.

8. The composition of claim 1, wherein the effective amount of curcumin is comprised of curcuminoids of about 92% to a range of about 94%.

9. The composition of claim 1, wherein the effective amount of ginger extract is gingerol in a range of about 10% to a range of about 12%.

10. The composition of claim 1, wherein the effective amount of vanilla extract is 0.5% to 2.5%.

11. The composition of claim 1, further comprising an effective amount of capsaicin.

12. The composition of claim 11, wherein the effective amount of capsaicin is 0.2 to 2%.

13. The composition of claim 1, further comprising pharmaceutically acceptable excipients.

14. The composition in claim 13, wherein the excipients are selected from a group consisting of granulating agents, binding agents, lubricating agents, disintegrating agents, sweetening agents, glidants, anti-adherents, anti-static agents, surfactants, antioxidants, coating agents, coloring agents, flavoring agents, plasticizers, preservatives, suspending agents, emulsifying agents and spheronization agents.

15. The composition in claim 1, wherein the composition is formulated into dosage forms selected from a group consisting of tablets, troches, lozenges, aqueous or oily suspensions, capsules, emulsions, creams, sprays, drops, dispersible powders or granules, syrups, elixirs, phytoceuticals, nutraceuticals and food stuffs.

16. A method of providing bioavailable curcumin to a mammal, the method comprising:
administering to a mammal in need thereof a composition comprising curcumin, and one or more items selected from the group consisting of vanilla extract, ginger extract and capsaicin.

17. The method of claim 16, further comprising administering pharmaceutically acceptable excipients to the mammal.

18. A composition for providing bioavailable curcumin, the composition comprising:
an effective amount of curcumin, and one or more selected from ginger extract, vanilla extract and capsaicin.

19. The composition of claim 1, wherein the effective amount of curcumin is comprised of curcuminoids of about 92% to a range of about 94%.

20. The composition of claim 1, wherein the effective amount of ginger extract is gingerol in a range of about 10% to a range of about 12%.

21. The composition of claim 1, wherein the effective amount of vanilla extract is 0.5% to 2.5%.

22. The composition of claim 1, further comprising 0.2 to 2% of capsaicin.

23. A method of reducing the size of one or more tumors in a mammal, the method comprising:
administering to a mammal having a tumor a composition containing an effective amount of curcumin, an effective amount of ginger extract and an effective amount of vanilla extract.
